(19) United States
(12) Patent Application Publication
(10) Pub. No.: US 2021/0291311 A1
(43) Pub. Date: Sep. 23, 2021

(54) METHOD FOR SIMULATING THE AGING OF A FABRIC

(71) Applicant: MANTECO S.P.A., Prato (IT)

(72) Inventors: Marco MANTELLASSI, Prato (IT); Matteo MANTELLASSI, Prato (IT); Franco MANTELLASSI, Prato (IT)

(21) Appl. No.: 17/196,157

(22) Filed: Mar. 9, 2021

(30) Foreign Application Priority Data
Mar. 17, 2020 (IT) 102020000005650

(51) Int. Cl. B24B 1/00; G01N 3/08; G01N 3/06; G01N 3/56

(52) U.S. Cl. CPC B24B 1/007; G01N 3/08; G01N 3/06; G01N 2203/0098; G01N 2203/0017; G01N 2203/0067; G01N 3/56

(57) ABSTRACT

Described is a method for simulating the ageing of a predetermined fabric comprising the following steps: preparing two specimens of said predetermined fabric, subjecting said specimens to an abrasion process by mutual rubbing, subjecting a first of said specimens to a test by means of an Elmendorf type lacerometer according to the direction of the weft of said predetermined fabric, subjecting a second of said specimens to an Elmendorf test according to the direction of warp of said predetermined fabric, comparing the results of said Elmendorf tests with a reference value relative to an Elmendorf test performed on the same predetermined fabric not subjected to any abrasion process.

This application claims priority to Italian Patent Application 102020000005650 filed Mar. 17, 2020, the entirety of which is incorporated by reference herein.

This invention relates to a method for simulating the ageing of a fabric.

The need is increasingly felt to carry out quality checks on fabrics which have just come out from the production step, that is to say, before they are manufactured into clothing or furnishing items.

The above-mentioned control is performed in such a way as to obtain an assessment of the durability of a predetermined fabric and of how it can withstand so-called ageing, due both to the passing of time and its use.

There are no prior art testing and measurement procedures designed to provide an overall quality value of the resistance to ageing of a fabric but only a multiplicity of individual tests designed to quantify certain mechanical, physical or chemical features.

For example, in order to obtain a laceration resistance value of the fabric, the so-called Elmendorf pendulum lacerometer is known to be used.

The Elmendorf pendulum makes it possible to perform a controlled operation of tearing the fabric in question by sliding a pendulum calibrated by weight.

Depending on the resistance provided by the fabric to obstruct movement of the pendulum, the instrument detects a value identifying the resistance to laceration of the fabric in question.

Another known instrument in the textile sector is the Martindale type abrasion meter.

This abrasion meter comprises a plurality of sectors rotating on respective contact supports.

It is also possible to set a contact pressure in the above-mentioned abrasive meter in order to increase or decrease the abrasive effect.

The Martindale abrasion meter is used to derive quantitative values of the resistance to abrasion of the fabric in question.

By using the Martindale abrasion meter it is possible to also quantify the so-called pilling resistance of the fabric, that is to say, the quantification of the surface formation of bobbles, which when entangled form small lumps.

There are also other methods or instruments but are all designed to provide a value independent from the others, that is to say, a function of a single characteristic whether it is of a mechanical-physical type (abrasion, laceration, pilling . . . ) or chemical type (colour migration, sweat fastness, washing fastness . . . ).

However, each single value obtained from the above-mentioned individual tests does not allow an overall qualitative value to be obtained which can be used as the simulation of the resistance to ageing of a fabric.

The term “ageing” means the wear and the transformations which a fabric can withstand during its life cycle, that is to say, the durability of the fabric.

The aim of the invention is therefore to provide a method for simulating ageing of a predetermined fabric which is able to provide an overall and qualitative value of a single fabric before it is manufactured into a final product.

The above-mentioned aim therefore allows the manufacturer to select in an optimised manner the individual fabric according to the duration for which the manufactured garment is designed.

A further aim of the invention is to provide a method for simulating the ageing of a predetermined fabric which can be replicated and is repeatable in a systematic manner and which therefore allows a reliable and sure value of the resistance to ageing of a fabric to be obtained.

The above aims are achieved by a method for simulating the ageing of a predetermined fabric comprising the technical features described in one or more of the accompanying claims.

Further features and advantages of the invention are more apparent in the detailed description below, with reference to a preferred, non-limiting, embodiment of method for simulating the ageing of a predetermined fabric.

The method for simulating the ageing of a predetermined fabric according to the invention comprises the following steps, in the following precise order.

A first step comprises preparing two specimens of the predetermined fabric and subjecting them to an abrasion process by mutual rubbing.

The expression “mutual rubbing” means a rubbing between two specimens of the same predetermined fabric being tested.

This step allows simulation of the rubbing which occurs during normal use of the manufactured finished garment, for example at the user's armpit or in any case where two flaps of fabric are subjected to mutual rubbing.

The method therefore comprises the step of subjecting a first of the two specimens already subjected to the mutual rubbing to a test by means of an Elmendorf lacerometer in the direction of the weft of the predetermined fabric, that is to say, positioning the specimen in the lacerometer in such a way that the tearing occurs in a direction perpendicular to the weft of the structure of the fabric under examination.

The method then comprises the step of subjecting the second of the two specimens to the same test by means of the Elmendorf pendulum abrasion meter, however this time in the direction of the warp of the predetermined fabric, that is to say, positioning the specimen in the lacerometer in such a way that tearing occurs in a direction perpendicular to the warp of the structure of the fabric under examination.

Advantageously, before carrying out the abrasion operation, the weft and warp directions are identified and marked on the reverse side of the two specimens.

Once the results of the above-mentioned two tests have been obtained, the results are compared with a reference value, both of the weft and warp tear, relative to a previous Elmendorf test performed on the same predetermined fabric but not subjected to any abrasion process, that is to say “new”.

Advantageously, the method according to the invention also comprises the steps of preparing a third specimen of the predetermined fabric and subjecting it to an abrasion process by rubbing against a reference abrasive fabric different from the fabric under examination.

Advantageously, the above-mentioned abrasion step takes place by applying a contact pressure of approximately 12 kPa and performing cycles of approximately 100,000 revolutions.

The method then comprises the step of subjecting the third specimen to a test using a Elmendorf pendulum type lacerometer and comparing the result of this test with a reference value relative to a previous test, again by means of an Elmendorf pendulum lacerometer, performed on a same predetermined fabric but not subjected to any abrasion process, that is to say “new”.

Advantageously, the above-mentioned abrasion step is performed by a Martindale type abrasion meter.

If the predetermined fabric under examination is of a mainly protein or mainly cellulosic nature, the method according to the invention comprises further steps.

The expression “fabrics of a mainly protein nature” is used to mean all those fabrics comprising a greater percentage of natural fibres of animal origin, such as, for example: wool, cashmere, silk . . . .

As mentioned, if, therefore, the predetermined fabric is of a mainly protein nature, the method also comprises the step of subjecting a fourth specimen of the fabric being examined to a bath in an aqueous solution with a pH of between 8 and 10, that is to say, an alkaline solution.

Advantageously, the fourth specimen is kept in a bath of the above-mentioned solution for 48 consecutive hours in such a way as to simulate the partial dissolving of the fibres of the fabric being examined.

Following the bath, the method comprises detecting some physical and dyeing features of the fourth specimen and, lastly, comparing them with the same physical and dyeing features detected on the same predetermined fabric not subjected to any process, that is to say “new”.

Alternatively, if the predetermined fabric under examination is of a mainly cellulosic nature, the method according to the invention comprises further steps.

The expression “mainly cellulosic fabrics” means all those fabrics comprising a greater percentage of natural fibres of vegetable origin, such as, for example: linen, cotton, hemp . . . .

As mentioned, if, therefore, the predetermined fabric is of a mainly cellulosic nature, the method also comprises the steps of subjecting a fourth specimen of the fabric being examined to a bath in an aqueous solution with a pH of between 3 and 5, that is to say, an acid solution.

Advantageously, the fourth specimen is kept in a bath of the above-mentioned solution for 48 consecutive hours in such a way as to simulate the emptying of the fibres of the fabric being examined.

Following the bath, the method comprises detecting some physical and dyeing features of the fourth specimen and, lastly, comparing them with the same physical and dyeing features detected on the same predetermined fabric not subjected to any process, that is to say “new”.

The term “physical features” means: tensile strength, laceration strength, seam creep strength, abrasion resistance and pilling resistance.

In detail:
the tensile strength value is measured by means of a dynamometer applied to an instrument which pulls a specimen of fabric until it breaks,
the laceration strength value is measured by means of a lacerometer, for example an Elmendorf pendulum,
the seam creep strength is measured by applying a dynamometer to two previously stitched flaps of the fabric in question,
the abrasion and pilling resistance values are measured by means of an abrasion meter, for example of the Martindale type.

The term “dyeing features” means: water fastness, sweat fastness, rubbing fastness, dry-cleaning fastness and washing fastness.

In detail:
water fastness is measured by placing a specimen of fabric under examination which was wetted previously in contact with a multifibre fabric for a predetermined time, for example 4 hours, and then measuring the quantity of colour migrated from one to the other,
sweat fastness is measured like water fastness, replacing the latter with a saline solution,
rubbing fastness is measured by rubbing the specimen of fabric under examination with a portion of cotton fabric for a predetermined time and then measuring the quantity of colour migrated from one to the other,
the washing fastness is measured by performing a series of normal washing cycles in water and solvent and
dry-cleaning fastness is measured by inserting the specimen of fabric under examination inside a container with cotton and metal portions to which a quantity of perchloroethylene is added.

The assessment of colour degradation is tested according to the ISO standard grayscale (from 1 to 5) whilst assessment of the change in appearance of the fabric (from 1 to 5) is tested according to AATCC 124 and subsequent modifications.

The wearing of the garment manufactured with the fabric under examination is also simulated, subjecting it to 5 cold tumble dryer cycles. The wearing of the garment is thus simulated by the beating of the fabric against the walls of the tumble dryer and by the rubbing against itself.

At the end of the test the following features are verified: comparative assessment with the original of the colour degradation, comparative assessment with the original of the appearance of the fabric. The method according to the invention brings important advantages.

An important advantage consists in the fact that the method according to the invention makes it possible to obtain an overall qualitative value of the resistance to ageing (from time and use) of a predetermined fabric.

The above-mentioned value is obtained by means of the weighted sum of the results of the above-mentioned individual tests so as to provide a single qualitative value for each individual fabric.

A further advantage achieved by the method according to the invention is that it allows the end user of the predetermined fabric, for example the manufacturer of the item of clothing, to be able to choose the fabric in a completely knowledgeable manner according to the actual final use of the item of clothing.

1. A method for simulating the ageing of a predetermined fabric comprising the following steps, in the precise order:
preparing two specimens of said predetermined fabric,
subjecting said specimens to an abrasion process by mutual rubbing,
subjecting a first of said specimens to a test by means of an Elmendorf type lacerometer in the direction of the weft of said predetermined fabric,
subjecting a second of said specimens to an Elmendorf test in the direction of the warp of said predetermined fabric,
comparing the results of said Elmendorf tests with a reference value relative to an Elmendorf test performed on the same predetermined fabric not subjected to any abrasion process,
preparing a third specimen of said fabric,
subjecting said third specimen to an abrasion process by rubbing against a reference abrasive fabric,
subjecting said third specimen to a laceration test using an Elmendorf pendulum abrasion meter,
comparing the result of said Elmendorf test with a reference value relative to an Elmendorf test performed on the same predetermined fabric not subjected to any abrasion process.

2. The method according to claim 1, wherein said step of subjecting said third specimen to an abrasion process by rubbing against a reference abrasive fabric occurs by applying a contact pressure of approximately 12 kPa and performing cycles of approximately 100,000 revolutions.

3. The method according to claim 1 comprising also, if said determined fabric is mainly protein in nature, the steps of:
subjecting a fourth specimen of said predetermined fabric to a bath in an aqueous solution with a pH of between 8 and 10,
detecting physical and dyeing features of said fourth specimen and
comparing said physical and dyeing features detected of said fourth specimen with the physical and dyeing features detected on the same predetermined fabric not subjected to any process.

4. The method according to claim 1, also comprising, if said predetermined fabric is of a mainly cellulosic nature, the steps of:
subjecting a fourth specimen of said predetermined fabric to a bath in an aqueous solution with a pH of between 3 and 5,
detecting physical and dyeing features of said fourth specimen and
comparing said physical and dyeing features detected of said fourth specimen with the physical and dyeing features detected on the same predetermined fabric not subjected to any process.

5. The method according to claim 2, wherein said physical features detected are: tensile strength, laceration strength, seam creep strength, abrasion resistance and pilling strength.

6. The method according to claim 5, wherein said tensile strength is measured by means of a dynamometer applied to an instrument which pulls one of said specimens of fabric until it breaks.

7. The method according to claim 4, wherein said seam creep strength is measured by means of a dynamometer applied to two previously stitched flaps of one of said specimens.

8. The method according to claim 2, wherein said dyeing features detected are:
water fastness, sweat fastness, rubbing fastness, dry-cleaning fastness and washing fastness.

9. The method according to claim 8, wherein said water fastness is measured by placing one of the specimens of the fabric previously wetted into contact with a multifibre fabric for a predetermined time and then measuring the quantity of colour migrated from said specimen to said multifibre fabric.

10. The method according to claim 7, wherein said rubbing fastness is measured by rubbing for a predetermined length of time one of said predetermined specimens of fabric with a portion of cotton fabric and then measuring the quantity of colour migrated from said specimen to said cotton portion.

11. The method according to claim 1, wherein said abrasion processes by rubbing are carried out by means of an abrasion meter of the Martindale type.

12. The method according to claim 1, also comprising the final step of providing a single qualitative value of said single predetermined fabric corresponding to the weighted sum of the results of said previous simulation steps.
